(19) United States
(12) Patent Application Publication
(10) Pub. No.: US 2007/0049585 A1
(43) Pub. Date: Mar. 1, 2007

(54) BENZAMIDE AND BENZOATE ANTI-HIV COMPOUNDS

(76) Inventors: Vassilios Papadopoulos, North Potomac, MD (US); Janet Greeson, Las Vegas, NV (US); Laurent Lecanu, McLean, VA (US)

Correspondence Address: SCHWEGMAN, LUNDBERG, WOESSNER & KLUTH, P.A., P.O. BOX 2938, MINNEAPOLIS, MN, 55402, US

(21) Appl. No.: 11/531,888

(22) Filed: Sep. 14, 2006

Related U.S. Application Data
(63) Continuation of application No. PCT/US05/09003, filed on Mar. 18, 2005.
(60) Provisional application No. 60/554,458, filed on Mar. 18, 2004.

(51) Int. Cl. A61K 31/54; A61K 31/5375; A61K 31/495; A61K 31/445; A61K 31/401; A61K 31/165; A61K 31/24

(52) U.S. Cl. 514/227.5; 514/237.5; 514/255.01; 514/317; 514/423; 514/619; 514/540

(57) ABSTRACT

The invention provides a therapeutic method for preventing or treating a pathological condition or symptom in a mammal, such as a human, wherein the infectivity of a pathogen such as a retrovirus toward mammalian cells is implicated and inhibition of its infectivity is desired comprising administering to a mammal in need of such therapy, an effective amount of procaine, procainamide or an analog thereof that inhibits pathogenic infectivity, including pharmaceutically acceptable salts thereof.

### RELATED APPLICATION

This application is a U.S. National Stage Application of PCT/US2005/009003 filed Mar. 18, 2005 and published in English as WO 2005/089453 on Sep. 29, 2005 which claims priority under 35 U.S.C. §119(e) from U.S. Provisional Application Ser. No. 60/554,458 filed Mar. 18, 2004, which applications and publication are herein incorporated by reference.

### BACKGROUND OF THE INVENTION

The global HIV/AIDS epidemic killed more than 3 million people in 2003, and an estimated 5 million acquired the human immunodeficiency virus (HIV)—bringing to 40 million the number of people living with the virus around the world. Despite progress in developing anti-viral regimens, there is not a fully effective therapy for AIDS. Current therapeutic strategies for AIDS include protease inhibitors, nucleoside analog reverse transcriptase inhibitors, non-nucleoside analog reverse transcriptase inhibitors, fusion inhibitors and also the highly toxic hydroxyurea (Yarchoan R et al. (1986) Lancet 1(8481): 575-580; Richards A D et al (1989) FEBS Lett 247(1): 113-117; Gao W Y et al. (1995) Proc Natl Acad Sci USA 92(18): 8333-8337; De Clercq E (1999) Farmaco 54(1-2): 26-45; Williams I G (2003) Int J Clin Pract 57(10): 890-897). Unfortunately, emerging resistances due to virus genotype mutations (Cavert W and Balfour H H (2003) Clin Lab Med 23(4): 915-928; Gallant J E et al. (2003) Antivir Ther 8(6): 489-506; Olson W C and Maddon P J (2003) Curr Drug Targets Infect Disord 3(4): 283-294) and serious side-effects are strong limitations to the treatment efficacy.

Currently, there is a need for effective anti-viral agents, including anti-retroviral agents. There is also a need for pharmacological tools for the further study of physiological processes associated with infection.

### SUMMARY OF THE INVENTION

The invention provides a method to prevent viral replication by blocking or inhibiting the ability of viruses, such as retroviruses, including HIV, to infect mammalian cells in vitro or in vivo. Thus, the present invention provides a method for treatment of a mammal exposed to an infectious pathogen including those threatened or afflicted by an infectious pathogen, such as a bacteria or virus, by administering to said mammal an effective amount of a compound of formula I: 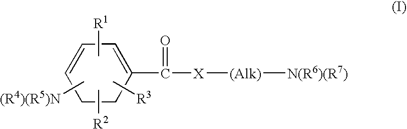
wherein:

a) R¹, R² and R³ are individually H, OH, halo, (C₁-C₆)alkyl, (C₁-C₆)alkoxy, (C₃-C₆)cycloalkyl, (C₃-C₆)cycloalkyl((C₁-C₆)alkyl), (C₂-C₆)alkenyl, (C₂-C₆)alkynyl, (C₁-C₆)alkanoyl, halo(C₁-C₆)alkyl, hydroxy(C₁-C₆)alkyl, (C₁-C₆)alkoxycarbonyl; (C₁-C₆)alkylthio or (C₁-C₆)alkanoyloxy; or R¹ and R² together are methylenedioxy;

b) R⁴, R⁵, R⁶ and R⁷ are individually, H, (C₁-C₆)alkyl, (C₃-C₆)cycloalkyl, (C₃-C₆)cycloalkyl((C₁-C₆)alkyl), (C₂-C₆)alkenyl, wherein cycloalkyl optionally comprises 1-2, S, nonperoxide O or N(R⁵); aryl, aryl(C₁-C₆)alkyl, aryl(C₂-C₆)alkenyl, heteroaryl, heteroaryl(C₁-C₆)alkyl, or R⁴ and R⁵ or R⁶ and R⁷ together with the N to which they are attached form a 5- or 6-membered heterocyclic or heteroaryl ring, optionally substituted with R¹ and optionally comprising 1-2, S, non-peroxide O or N(R⁵);

c) (Alk) is (C₂-C₆)alkyl, (C₂-C₆)alkenyl, (C₃-C₆)cycloalkyl, (C₃-C₆)cycloalkyl(C₂-C₆)alkyl or [(C₂-C₆)alkyl(C₃-C₆)cycloalkyl[(C₃-C₆)alkyl] optionally substituted by 1-2 S, non-peroxide O or N(R⁵); and

d) X is O or NH;

and the pharmaceutically acceptable salts thereof.

Preferably (Alk) is (C₂-C₄)alkyl, such as —(CH₂)₂—, —(CH₂)₃— or —(CH₂)₄—.

Preferably, both of R⁴ and R⁵ is H.

Preferably, both R⁶ and R⁷ are (C₁-C₆)alkyl or (C₃-C₆)cycloalkyl.

Preferably, 1 or 2 of R¹, R² or R³ is (C₁-C₆)alkoxy.

Preferably, (R⁵)(R⁴)N— is in the para or 4-position.

The invention also provides a pharmaceutical composition comprising a compound of formula I, or a pharmaceutically acceptable salt thereof, in combination with a pharmaceutically acceptable diluent or carrier, which optionally can include one or more anti-HIV agents of one or more of the classes of anti-HIV agents referenced herein above, and can optionally include stabilizers, preservatives, and absorption control agents.

Additionally, the invention provides a therapeutic method for preventing or treating a pathological condition or symptom in a mammal, such as a human, wherein the infectivity of a pathogenic agent or microorganism such as a retrovirus toward mammalian cells is implicated and inhibition of its infectivity is desired comprising administering to a mammal in need of such therapy, an effective amount of a compound of formula I, or a pharmaceutically acceptable salt thereof.

The invention provides a compound of formula I for use in medical therapy (e.g., for use in treating a mammal infected, e.g., with a retrovirus such as HIV), as well as the use of a compound of formula I for the manufacture of a medicament useful for the treatment of infection in a mammal, such as a human.

The invention also provides a method for binding a compound of formula I to mammalian cells to alter the permeability of cell walls to infectious agents comprising contacting the cells in vivo or in vitro, with an amount of a compound of formula I effective to interact with, and to alter the properties of the walls of said cells, e.g., to alter the sterol composition of the cell walls. Cells comprising a compound of formula I as a ligand bound to receptor sites can be used to measure the selectivity of test compounds for specific receptors on or in cell walls, or can be used as a tool to identify potential therapeutic agents for the treatment of diseases or conditions dependent on cell wall permeability, by contacting said agents with said ligand-receptor complexes, and measuring the extent of displacement of the ligand and/or binding of the agent.

The invention also provides novel compounds of formula I, as well as, processes and intermediates disclosed herein that are useful for preparing compounds of formula (I) or salts thereof

### BRIEF DESCRIPTION OF THE FIGURES

FIG. 1 depicts the chemical structure of SP01 and SP100. SP010 is a complex procainamide derivative that is not within the scope of the compounds of formula I.

FIG. 2, panels A-C are graphs depicting the inhibitory effect of SP01, SP010 and SP100 on the HIV-1 IIIB strain replication in HeLa cells. Compounds were tested either alone or in a formulation (1A, 010A or 100A). 3TC, ddl and AZT are known anti-viral compounds.

FIG. 3, panels A-C are graphs depicting the inhibitory effect of 24-hour SP01, SP010 and SP100 premedication on the HIV-1 IIIB strain replication in HeLa cells. Compounds were tested in a formulation (01A, 010A or 100A).

FIG. 4, panels A-C are graphs depicting the inhibitory effect of 48-hour SP01, SP010 and SP100 premedication on the HIV-1 IIIB strain replication in HeLa cells.

FIG. 5, panels A-C are graphs depicting the inhibitory effect of SP01 , SP01A and SP010 on the multi-drug resistant HIV MDR-769 strain replication in HeLa cells.

### DETAILED DESCRIPTION

The following definitions are used, unless otherwise described: halo is fluoro, chloro, bromo, or iodo. Alkyl, alkoxy, alkenyl, alkynyl, etc. denote both straight and branched groups; but reference to an individual radical such as Apropyl@ embraces only the straight chain radical, a branched chain isomer such as Aisopropyl@ being specifically referred to. Aryl denotes a phenyl radical or an ortho-fused bicyclic carbocyclic radical having about nine to ten ring atoms in which at least one ring is aromatic. Heteroaryl encompasses a radical attached via a ring carbon of a monocyclic aromatic ring containing five or six ring atoms consisting of carbon and one to four heteroatoms each selected from the group consisting of non-peroxide oxygen, sulfur, and N(X) wherein X is absent or is H, O, (C₁-C₄)alkyl, phenyl or benzyl, as well as a radical of an ortho-fused bicyclic heterocycle of about eight to ten ring atoms derived therefrom, particularly a benz-derivative or one derived by fusing a propylene, trimethylene, or tetramethylene diradical thereto.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase) and how to determine anti-infectious activity using the standard tests described herein, or using other similar tests which are well known in the art.

Specific and preferred values listed below for radicals, substituents, and ranges, are for illustration only; they do not exclude other defined values or other values within defined ranges for the radicals and substituents.

Specifically, (C₁-C₆)alkyl can be methyl, ethyl, propyl, isopropyl, butyl, iso-butyl, sec-butyl, pentyl, 3-pentyl, or hexyl; (C₃-C₆)cycloalkyl can be cyclopropyl, cyclobutyl, cyclopentyl, or cyclohexyl; (C₃-C₆)cycloalkyl(C₁-C₆)alkyl can be cyclopropylmethyl, cyclobutylmethyl, cyclopentylmethyl, cyclohexylmethyl, 2-cyclopropylethyl, 2-cyclobutylethyl, 2-cyclopentylethyl, or 2-cyclohexylethyl; heterocycloalkyl and heterocycloalkylalkyl includes the foregoing cycloalkyl wherein the ring optionally comprises 1-2 S, non-peroxide O or N(R⁵) as well as 2-5 carbon atoms; such as morpholinyl, piperidinyl, piperazinyl, indanyl and the like; (C₁-C₆)alkoxy can be methoxy, ethoxy, propoxy, isopropoxy, butoxy, iso-butoxy, sec-butoxy, pentoxy, 3-pentoxy, or hexyloxy; (C₂-C₆)alkenyl can be vinyl, allyl, 1-propenyl, 2-propenyl, 1-butenyl, 2-butenyl, 3-butenyl, 1,-pentenyl, 2-pentenyl, 3-pentenyl, 4-pentenyl, 1-hexenyl, 2-hexenyl, 3-hexenyl, 4-hexenyl, or 5-hexenyl; (C₂-C₆)alkynyl can be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl; (C₁-C₆)alkanoyl can be formyl, acetyl, propanoyl or butanoyl; halo(C₁-C₆)alkyl can be iodomethyl, bromomethyl, chloromethyl, fluoromethyl, trifluoromethyl, 2-chloroethyl, 2-fluoroethyl, 2,2,2-trifluoroethyl, or pentafluoroethyl; hydroxy(C₁-C₆)alkyl can be hydroxymethyl, 1-hydroxyethyl, 2-hydroxyethyl, 1-hydroxypropyl, 2-hydroxypropyl, 3-hydroxypropyl, 1-hydroxybutyl, 4-hydroxybutyl, 1-hydroxypentyl, 5-hydroxypentyl, 1-hydroxyhexyl, or 6-hydroxyhexyl; (C₁-C₆)alkoxycarbonyl can be methoxycarbonyl, ethoxycarbonyl, propoxycarbonyl, isopropoxycarbonyl, butoxycarbonyl, pentoxycarbonyl, or hexyloxycarbonyl; (C₁-C₆)alkylthio can be methylthio, ethylthio, propylthio, isopropylthio, butylthio, isobutylthio, pentylthio, or hexylthio; (C₂-C₆)alkanoyloxy can be acetoxy, propanoyloxy, butanoyloxy, isobutanoyloxy, pentanoyloxy, or hexanoyloxy; aryl can be phenyl, indenyl, or naphthyl; and heteroaryl can be furyl, imidazolyl, triazolyl, triazinyl, oxazoyl, isoxazoyl, thiazolyl, isothiazoyl, pyrazolyl, pyrrolyl, pyrazinyl, tetrazolyl, pyridyl, (or its N-oxide), thienyl, pyrimidinyl (or its N-oxide), indolyl, isoquinolyl (or its N-oxide) or quinolyl (or its N-oxide).

The term “retrovirus” includes, but is not limited to, the members of the family retroviridae, including alpharetroviruses (e.g., avian leukosis virus), betaretroviruses (e.g., mouse mammary tumor virus), gammaretroviruses (e.g., murine leukemia virus), deltaretroviruses (e.g., bovine leukemia virus), epsilonretroviruses (e.g., Walley dermal sarcoma virus), lentiviruses (e.g., HIV-1) and spumaviruses (e.g., human spumavirus).

Benzoates useful in the present invention comprise a number of the topical anesthetics, which are an art-recognized class of drugs which temporarily interrupt mammalian nerve transmissions. They can generally be grouped into two chemical classifications structurally; the N-arylamides or carboxamides, such as lidocaine; and the aminoalkylbenzoates such as procaine, tetracaine, benoxinate and proparacaine.

The aminoalkylbenzoates include esters between benzoic acids and alcohols of the general formula (R⁶)(R⁷)N(Alk)OH, wherein Alk is as defined above. R⁶ is H or (C₁-C₄)-alkyl, R⁷ is (C₁-C₄)alkyl or R⁶ and R⁷ taken together with N are a 5- or 6-membered heterocycloaliphatic ring, optionally substituted by (C₁-C₃)alkyl or comprising an additional ring O— or N-atom. The benzoic acid moiety can be the moiety (R⁸)(R⁹)ArCO₂H wherein Ar is an aromatic —C₆H₂₋₄— radical “phenylene” and each R⁸ and R⁹ is H, halo, preferably Cl, (R⁵)(H)N—, H₂N— or (C₁-C₅)alkoxy.

Useful topical anesthetics including chloroprocain (4-amino-2-chlorobenzoic acid 2-(diethylamino)ethyl ester); procaine (4-aminobenzoic acid 2-(diethylamino)ethyl ester); tetracaine (4-(butylamino)benzoic acid 2-(dimethylaminoethyl ester; see Shupe (U.S. Pat. No. 3,272,700)); benoxinate (4-amino-3-butoxybenzoic acid 2-(diethylamino)ethyl ester (U.K. Patent No. 654,484)) proparacaine (3-amino-4-propoxybenzoic acid 2-(diethylamino)ethyl ester); isobucain (1-propanol, 2-methyl-2-[(2-methylpropyl)amino]benzoate; meprylcaine ([(2-methyl)(2-propylamino)propyl]benzoate; piperocaine ((2-methylpiperidin-1-ylpropyl(benzoate)); propoxycaine (2-(diethylamino)ethyl-([2′-methyl-4-amino]benzoate)); butacaine (((3-dibutylamio)propyl)-(2′-aminobenzoate)); cyclomethylcaine (((3-2′-methylpiperidine-1-yl))propyl)-[4′-cyclohexyloxy-benzoate]); hexylcaine (([2-cyclohexylamino)(1-methyl)]ethyl)(benzoate) and proparacaine (((2-diethylamino)ethyl)[(4′-propyloxy-3′-amino)benzoate]).

A specific value for R¹ in formula I, above is H, (C₂-C₄)alkyl, (C₂-C₄)alkoxy or (C₃-C₆)heterocycloalkyl.

A specific value for R² is H.

A specific value for R³ is H.

A specific value for N(R⁴)(R⁵) is amino.

A specific value for N(R⁶)(R⁷) is diethyl amino, dipropylamino, cyclohexylamino, or propylamino.

A specific value for (Alk) is —(CH₂)₂₋₃—.

A specific value for X is O.

A preferred group of compounds are compounds of formula I which are aminoalkyl benzoates.

Another preferred group of compounds are compounds of formula I which are N-aminoalkyl-benzamides.

A preferred compound of the invention is a procaine or procainamide, or an analog thereof.

In cases where compounds are sufficiently basic or acidic to form stable nontoxic acid or base salts, administration of the compounds as salts may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium), alkaline earth metal (for example calcium or magnesium) or zinc salts can also be made.

One embodiment of the present invention provides a composition including a compound of formula I and a zinc salt, such as zinc sulfate heptahydrate, wherein ascorbic acid is not preferred in the composition due to a browning effect, e.g., degradation of one or more of the components. In one embodiment, a compound of formula I and a zinc salt, e.g., zinc sulfate heptahydrate, are present in a composition at a ratio of about 27-107 to 1.

The compounds of formula I can be formulated as pharmaceutical compositions and administered to a mammalian host, such as a human patient in a variety of forms adapted to the chosen route of administration, i.e., orally or parenterally, by intravenous, intramuscular, topical or subcutaneous routes, or by inhalation or insufflation.

Thus, the present compounds may be systemically administered, e.g., orally, in combination with a pharmaceutically acceptable vehicle such as an inert diluent or an assimilable edible carrier. They may be enclosed in hard or soft shell gelatin capsules as powders, pellets or suspensions or may be compressed into tablets. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices, such as patches, infusion pumps or implantable depots.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection, infusion or inhalation can include sterile aqueous solutions or dispersions. Sterile powders can be prepared comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate, cellulose ethers, and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of formula I to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compounds of formula I can be determined by comparing their in vitro activity, and in vivo activity in animal models. Methods for the extrapolation of effective dosages in mice, and other animals, to humans are known to the art; for example, see U.S. Pat. No. 4,938,949.

Generally, the concentration of the compound(s) of formula I in a liquid composition, such as a lotion, will be from about 0.1-25 wt-%, preferably from about 0.5-10 wt-%. The concentration in a semi-solid or solid composition such as a gel or a powder will be about 0.1-5 wt-%, preferably about 0.5-2.5 wt-%.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently administered in unit dosage form; for example, containing 5 mg to as much as 1-3 g, conveniently 10 to 1000 mg, most conveniently, 50 to 500 mg of active ingredient per unit dosage form.

Ideally, the active ingredient should be administered to achieve peak plasma concentrations of the active compound of from about 0.5 to about 75 μM, preferably, about 1 to 50 μM, most preferably, about 2 to about 30 μM. This may be achieved, for example, by the intravenous injection of a 0.05 to 5% solution of the active ingredient, optionally in saline. For example, as much as about 0.5-3 g of a compound of formula I can be dissolved in about 125-500 ml of an intravenous solution comprising, e.g., 0.9% NaCR, and about 5-10% glucose. Such solutions can be infused over an extended period of up to several hours, optionally in conjunction with other anti-viral agents, antibiotics, etc. The active ingredient can also be orally administered as a bolus containing about 1-100 mg of the active ingredient. Desirable blood levels may be maintained by continuous infusion to provide about 0.01-5.0 mg/kg/hr or by intermittent infusions containing about 0.4-15 mg/kg of the active ingredient(s).

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The ability of a compound of the invention to act as an antiviral agent may be determined using pharmacological models which are well known to the art, or using tests described below.

The following illustrate representative pharmaceutical dosage forms, containing a compound of formula I, for therapeutic or prophylactic use in humans.

| (i) Tablet 1 | mg/tablet |
|---|---|
| SP01 or SP100 | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |
| (ii) Tablet 2 | mg/tablet |
| SP01 or SP100 | 20.0 |
| Microcrystalline cellulose | 410.0 |
| Starch | 50.0 |
| Sodium starch glycolate | 15.0 |
| Magnesium stearate | 5.0 |
| | 500.0 |
| (iii) Capsule | mg/capsule |
| SP01 or SP100 | 10.0 |
| Colloidal silicon dioxide | 1.5 |
| Lactose | 465.5 |
| Pregelatinized starch | 120.0 |
| Magnesium stearate | 3.0 |
| | 600.0 |
| (iv) Injection 1 (1 mg/ml) | mg/ml |
| SP01 or SP100 (free acid form) | 1.0 |
| Dibasic sodium phosphate | 12.0 |
| Monobasic sodium phosphate | 0.7 |
| Sodium chloride | 4.5 |
| 1.0 N Sodium hydroxide solution | q.s. |
| (pH adjustment to 7.0-7.5) | |
| Water for injection | q.s. ad 1 mL |
| (v) Injection 2 (10 mg/ml) | mg/ml |
| Attorney docket no.: 1941.004US2 | 13 |
| SP01 or SP100 (free acid form) | 10.0 |
| Monobasic sodium phosphate | 0.3 |
| Dibasic sodium phosphate | 1.1 |
| Polyethylene glycol 400 | 200.0 |
| 01 N Sodium hydroxide solution | q.s. |
| (pH adjustment to 7.0-7.5) | |
| Water for injection | q.s. ad 1 mL |
| (vi) Aerosol | mg/can |
| SP01 or SP100 | 20.0 |
| Oleic acid | 10.0 |
| Trichloromonofluoromethane | 5,000.0 |
| Dichlorodifluoromethane | 10,000.0 |
| Dichlorotetrafluoroethane | 5,000.0 |

The above formulations may be prepared by conventional procedures well known in the pharmaceutical art.

The invention will be further described by reference to the following detailed examples.

### EXAMPLE 1

### In Vitro Study of the Inhibition of HIV-1 IIIB Replication on HeLa Cells by Procaine and Procaine Derivatives

A. Methods

In order to study the viral replication in vitro, the GenPhar (Mt. Pleasant, S.C.) AV-Finder™-HIV Drug Discovery Assay was used, that consists of two components: (1) a cloned, continuous-passage HeLa cell line containing an HIV-1 tat-activated molecular switch and a Green Fluorescent Protein reporter gene and (2) a recombinant adenovirus (rAd) vector containing the genes for all three of the HIV-1 receptor/co-receptors (CD4, CXCR4, and CCR5) to transduce into HeLa cells and convert them into highly susceptible HIV-1 indicator cells for use in the assay. The indicator cells over-express the HIV-1 receptor genes and are readily infected with any HIV-1 strain or isolate. All HIV-1 strains tested thus far, regardless of co-receptor preference, and all subtypes or clades of HIV-1 will infect these indicator cells. Infected cells fluoresce brightly so that the inhibition of virus replication by potential antiviral drugs can be readily detected and quantified using standard laboratory plate reader technology.

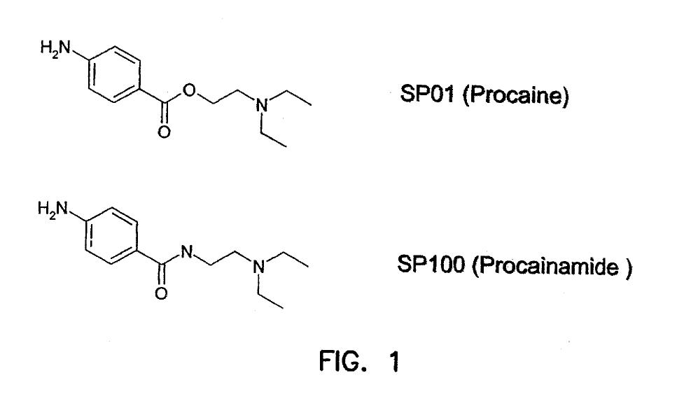

Detector plates are set up at day 1 by adding HeLa cells (3000/well) to the adenovirus AD-3R in DMEM containing CCS in 96-well plates and to incubate at 37° C. under 95% humidity and 5% CO₂ for 2 days. Without pre-medication, at day 3, HIV-1 IIIB (200 IP/well) and increasing concentrations of procaine, procainamide (both from Aldrich-Sigma), SP10, or reference compounds (AZT, ddI, 3TC) were added and incubated overnight. At day 4, the medium was replaced by fresh medium containing the corresponding concentration of the compounds of interest. The infectivity was assessed by measuring the fluorescence on each well at day 7 (λₑₘᵢₛ=485 nm; λ_{exc}=520 nm). With 24 hours pre-medication, increasing concentrations of procaine, procainamide, SP10 (FIG. 1) or reference compounds (AZT, ddI, 3TC) were added at day 3 and incubated overnight. At day 4, HIV-1 IIIB (200 IP/well) and increasing concentrations of procaine, procainamide, SP10 or reference compounds (AZT, ddI, 3TC) were added and incubated overnight. At day 5, the medium was replaced by fresh medium containing the corresponding concentration of compounds of interest and the infectivity was assessed by measuring the fluorescence on each well at day 8. Results are expressed as percentage of inhibition of the viral replication.

Following the above described cell treatment protocol, the levels of cellular 3-(4,5-dimethylthiazol-2-yl)-2,5-diphenyl tetrazolium bromide (MTT) reduction, a measure of mitochondrial integrity, were determined in order to examine whether the compounds tested were cytotoxic.

Procaine HCl was used either alone dissolved in water (SP01) or in an Anticort-like formulation (SP01A) containing zinc sulfate heptahydrate and ascorbic acid at the ratio of about 27-107/1/1.3-2.0 (for example 200 mg procaine HCl with 7.5 mg of zinc sulfate heptahydrate and 12.5 mg of ascorbic acid; Xu, J. et al. J Pharmacol. Exper. Ther. 2003 307:1148-1157) (Samaritan Pharmaceuticals).

B. Results

1. Effect on HIV-1 IIB Viral Replication. No Pre-Medication.

The structures of the compounds procaine HCl (SP01) and procainamide (SP100) are shown in FIG. 1. Compounds were dissolved in water or when indicated in the Anticort-like formulation (SP01A, SP100A, SP10A).

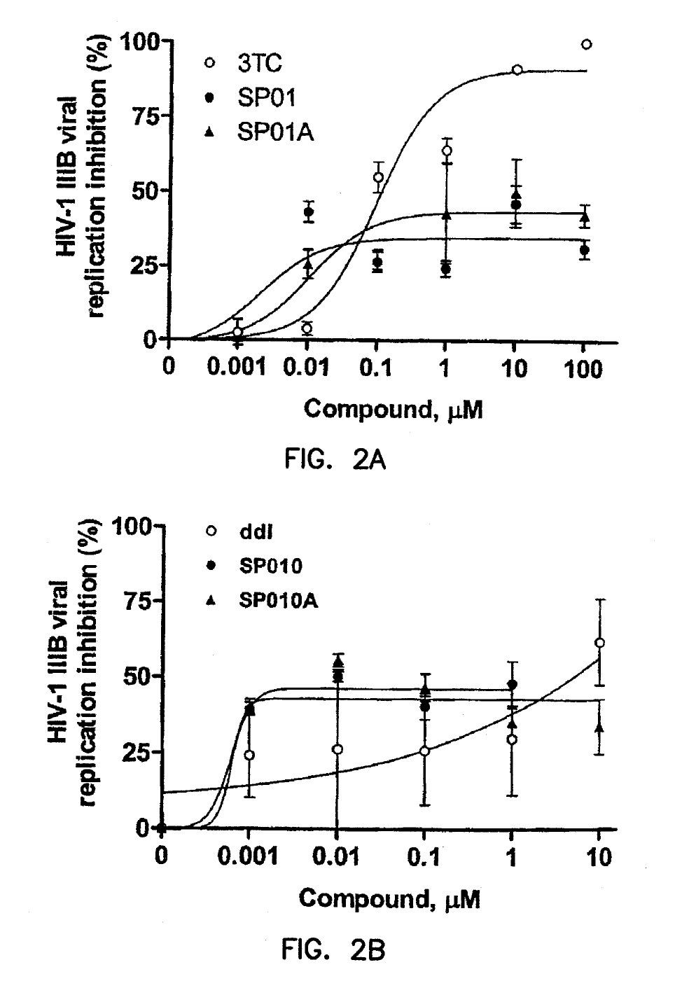

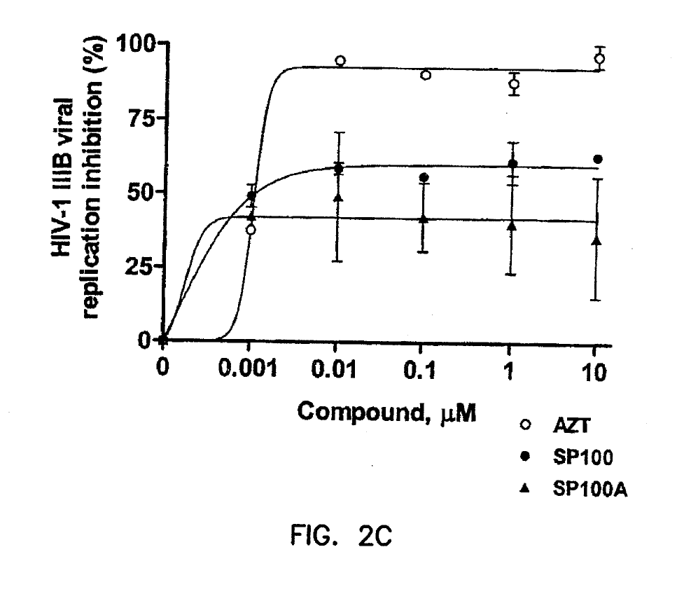

SP01 inhibited the HIV-1 IIIB viral replication with a higher efficacy than the classical antiviral agent 3TC when used at concentrations up to 0.1 μM (FIG. 2A). SP01 A also inhibited viral replication in a dose-dependent manner reaching a 43% inhibition compared to 90% inhibition obtained with maximal concentrations of 3TC (FIG. 2A). Interestingly SP01 and SP01A at all concentrations tested, up to 100 μM, were devoid of cell toxicity as assessed by the MTT cytotoxicity assay, in contrast to 3TC which showed toxicity with an IC50 of 71 μM. In further studies, the antiviral agents ddI and AZT were found to be cytotoxic with IC50s of 89 and 161 μM concentrations, respectively. Thus, in order to be able to accurately compare the antiviral properties of the compounds under investigation to that of classical antiviral agents, concentrations ranging from pM up to 10 μM were used. SP10 and SP10A were found to be more potent that ddI at concentrations up to 1 μM (FIG. 2B), inhibiting viral replication by 40%. For both SP10 and SP10A the strongest inhibition was observed at 0.01 μM inhibiting by 55.60±2.12% and 50.20±1.70% (p(>0.001), respectively, viral replication compared to 26.37±26.11% (p<0.05) inhibition observed by ddI.

2. Effect on HIV-1 IIB Viral Replication. Effects of 24 Hours Pre-Medication.

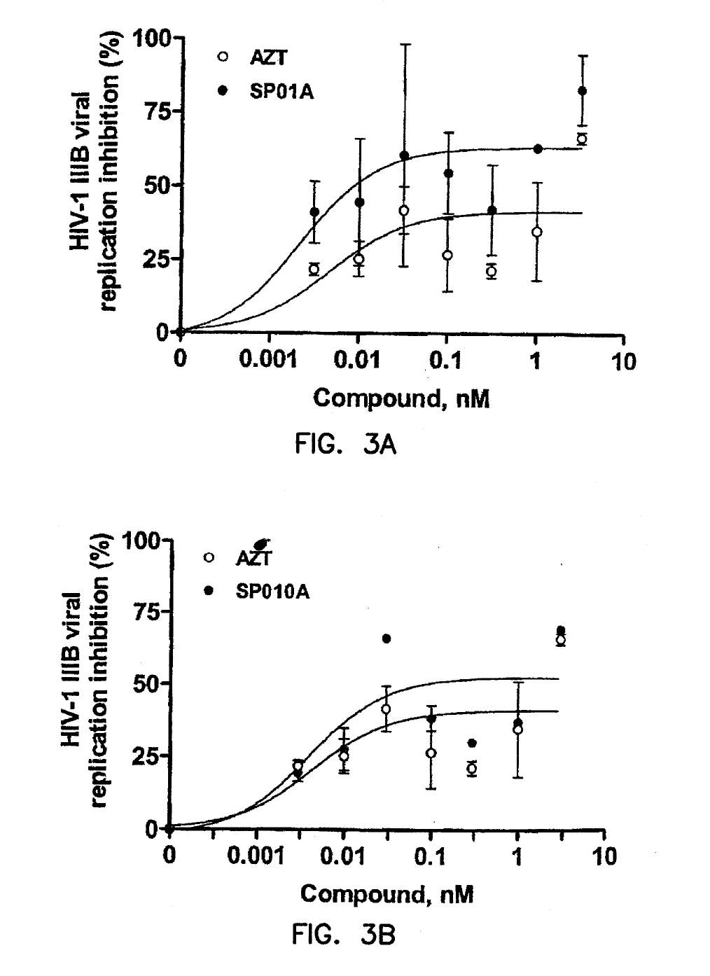

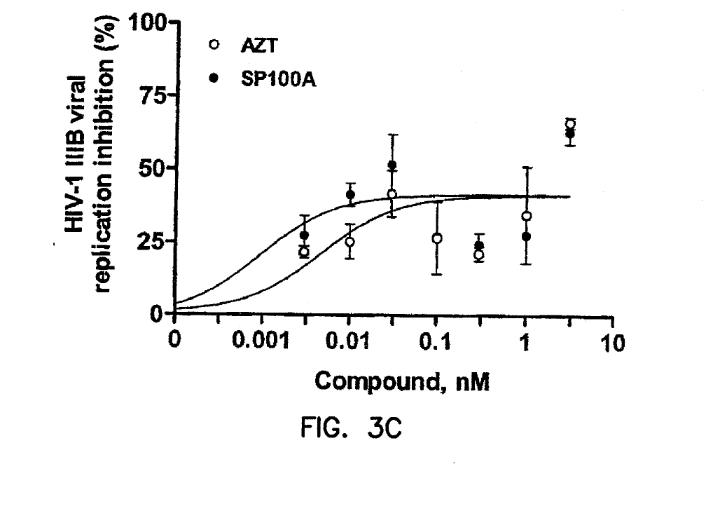

Except for AZT, all the compounds tested were dissolved in the Anticort-like solution. After 24 hours pre-medication, all of them displayed at a better efficacy than AZT on the viral replication (FIG. 3). SP01A (FIG. 3A) and SP010A (FIG. 3B) reduced viral replication in a more dramatic manner compared to AZT reaching a plateau of 63% and 52% inhibition for SP01A and SP10A respectively, compared to 32% inhibition by AZT. The peak of the inhibitory activity observed was 0.03 nM for SP01A and SP010. SP100 was also effective, but at the same extent as AZT (FIG. 3C).

3. Effect on HIV-1 IIB Viral Replication. Effects of 48 Hours Pre-Medication.

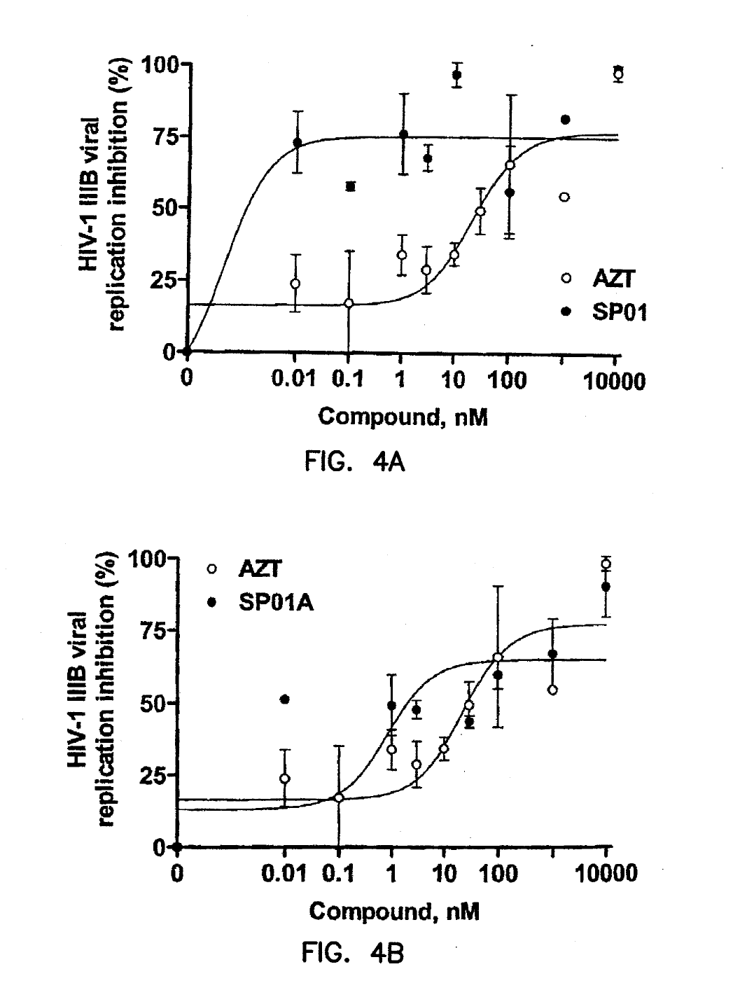

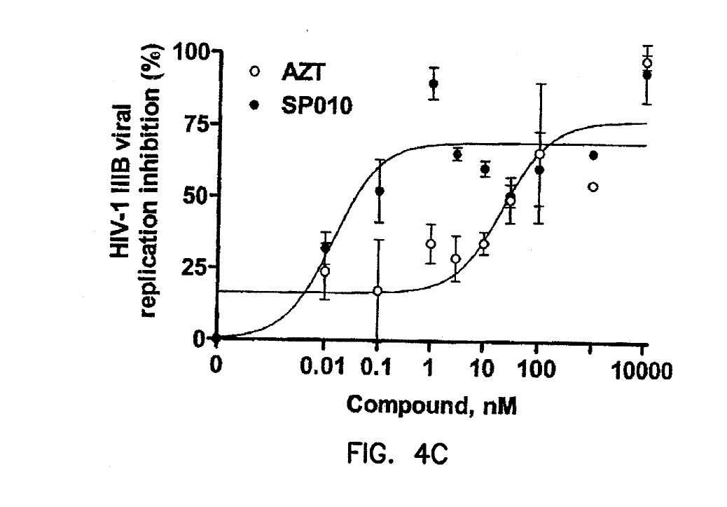

Forty-eight hours pretreatment with SP01 inhibited by 75% HIV replication at all concentrations tested (FIG. 4A). Under the same protocol AZT inhibited the HIV replication in a dose-dependent manner with an IC50 of 30 nM. 48 hours pretreatment with SP01A also inhibited viral replication (FIG. 4B) and the same was true for SP010 which inhibited with an IC50 of 0.01 nM (FIG. 4C).

4. Effect on HIV MDR 769 Viral Replication. Effects Without Pre-Medication.

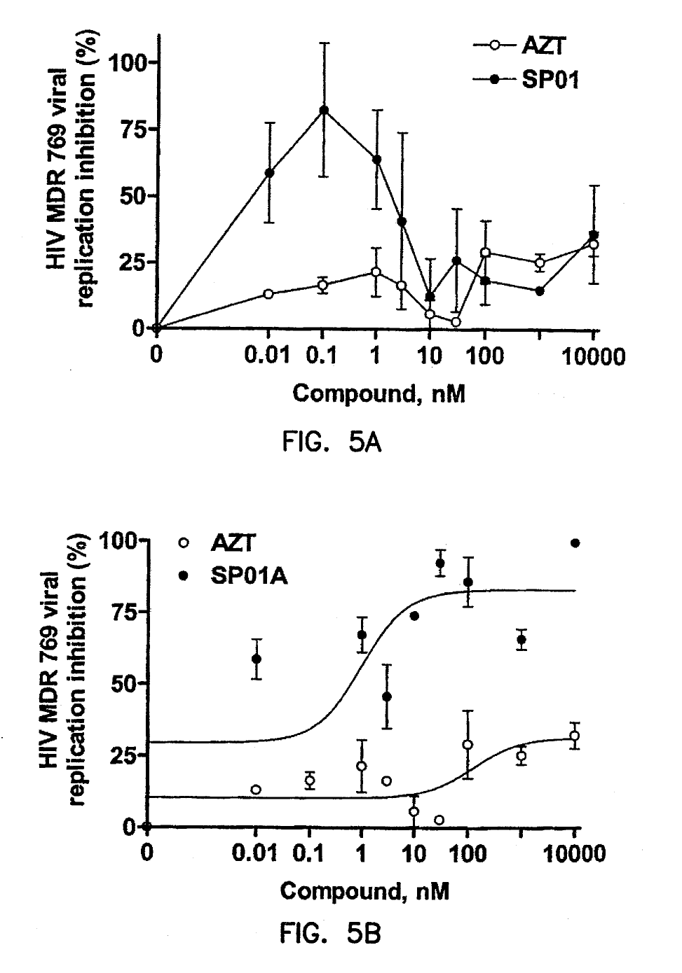

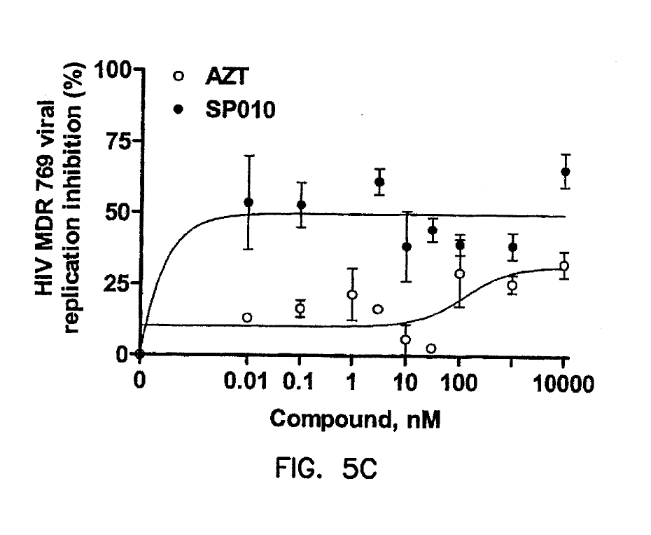

As expected AZT was not effective in inhibiting the HIV MDR 769 strain replication (FIGS. 5A, B, C). SP01 inhibited by 75% the HIV MDR 769 viral replication at concentrations up to 1 nM. At higher concentrations the compound was not effective. In contrast SP01A effectively inhibited the MDR HIV strain replication at all concentrations tested, reaching up to 80% inhibition. SP010 also inhibited the replication of the MDR HIV strain although with a maximal efficacy reaching 50%.

### EXAMPLE 2

### Clinical Study

A. Methodology

1. Ethical Conduct of the Study

This study was conducted in accordance with ethical principals that are consistent with good clinical practice and applicable regulatory requirements.

2. Study Drug and Doses Administered

Capsules of 200 mg Procaine HCl were supplied by Samaritan Pharmaceuticals in a formulation containing procaine HCl, zinc sulfate heptahydrate (to decrease the rate of absorption of procaine), ascorbic acid (as an antioxidant), potassium benzoate, and disodium phosphate and sodium sorbate as a preservative. The dose was determined by prior studies of the bioavailability of procaine HCl and the doses used in previous studies of procaine HCl in the treatment of depression in elderly persons (Whalen et al. J. Clin. Epidemiol. 1994 47: 537-546; Cohen et al., Psychosomatics 1974 15: 15-19; Sakalis et al. Current Therapeutic Research 1974 16: 59-63).

3. Selection of Study Population

Eligible patients were ≧18 years, HIV-1 positive (cohorts A, B, C, D); on stable triple antiretroviral regimen for the preceding 8 weeks; with current CD4 counts>200/mm³.

4. Study Design

The study was a non-randomized, Phase II, open-label, single investigative center, eight-week study sequentially using four doses of orally administered procaine HCl: 200 mg (cohort A), 400 mg (cohort B), 600 mg (cohort C) and 800 mg (cohort D). Six subjects were enrolled per cohort. During the screening phase of the study, subjects previously diagnosed with HIV-1 provided written informed consent. Each potential participant underwent complete medical history, and all medications taken within the past 3 months and any current medications were reviewed. Each potential participant underwent clinical laboratory tests, including RNA PCR to determine viral load as well as infection screening (HIV antibody test).

Patients returned on Day 7 to begin the 8 weeks of medication administration. They were given daily medication diaries to record when they are taking their study medication. Subjects underwent complete clinical and biological examinations. HIV negative subjects were discharged, having completed their part of the study. In the subsequent visits of weeks 2, 3, 4, 6, 9 (last dose of medication), each subject underwent clinical laboratory tests, including viral load by NASBA. Patients received their last dose of medication on day 64. Patients returned at week 11 (end of study) for complete laboratory tests.

5. Efficacy Variables: Viral Load Measurements

Viral load was measured by NASBA Assay (Using Nuclisens assay from Organon Technica®) with a lower limit of detection of 50 copies/ml, banked samples were stored at −70° C.

6. Statistical Methods

For each dose level (A-D), changes (week 9-baseline) in efficacy variables were tested for significance using a paired Student t-test (two sided). Analyses of variance (ANOVA) and analyses of covariance (ANCOVA) were conducted to compare the changes in safety and efficacy (covariate=baseline values) variables across the four dose levels, respectively. In addition, regression analyses were conducted to test for a linear trend in efficacy variables across the four dose levels. Changes from baseline to week 9 for all four dose levels combined were tested using paired t-tests. Similar analyses were conducted for changes from week 9 to week 11 to assess potential “rebound effects” after the drug was removed. Mixed effects modeling procedures were used to test for linear and quadratic trends across all study visits. Finally, subgroup analyses which combined low vs. high dose levels were also conducted. The significance level was set at 0.05. Statistical analyses utilized SAS v9.0 (Carey, N.C.).

The results obtained in vitro were analyzed by ANOVA followed by a Dunnett's test.

7. Demographics

30 male patients entered the study, of whom 24 received procaine HCl; there were 12 Caucasian, 7 Hispanic, 9 black, 1 Asian, 1 self-defined as “other.” Mean age was of 42 (38-49) years Cohort A, 46 (39-52) cohort B, 40 (34-60) cohort C and 42 (37-52) cohort D, years. All subjects completed the protocol but one (cohort A) who left the study on day 7 after receiving one dose of study drug and was not replaced.

B. Efficacy Evaluation

1. Viral Load (Table 1)

Because the subjects in the study had to be on HAART, the majority of subjects entered with undetectable viral load measures. But for the patients in the study with detectable viral loads, viral load measures tended to decrease over time. In an attempt to obtain additional measures of viral load changes, stored samples from patients who had undetectable viral loads were run using the more sensitive FDA approved NASBA assay which has a lower limit of detection (50 copies/ml). Results from these assays are shown in Table 1.

TABLE 1
Mean Changed Values Across Cohort and All cohort combined in Viral Load

| | | | | | | | | | | | | | Linear |
|---|---|---|---|---|---|---|---|---|---|---|---|---|---|
| Cohort A | | | Cohort B | | | Cohort C | | | Cohort D | | | Cohort | Trend |
| Mean | SD | P* | Mean | SD | P* | Mean | SD | P* | Mean | SD | P* | P-value** | P-value |

A. From Baseline to Week 9

| | | | | | | | | | | | | | | |
|---|---|---|---|---|---|---|---|---|---|---|---|---|---|---|
| Viral Load^{H} | −0.52 | 0.98 | 0.30 | −0.21 | 0.65 | 0.51 | −0.79 | 0.42 | 0.03 | −0.54 | 1.46 | 0.41 | 0.23 | 0.78 |
| 2 patients omitted from | −0.64 | 2.15 | 0.60 | 0.48 | 1.49 | 0.51 | −1.82 | 0.97 | 0.03 | −0.10 | 2.10 | 0.92 | 0.40 | 0.87 |
| analysis | | | | | | | | | | | | | | |

B. From Week 9 to Week 11

| | | | | | | | | | | | | | | |
|---|---|---|---|---|---|---|---|---|---|---|---|---|---|---|
| Viral Load^{H} | −0.48 | 0.61 | 0.21 | −0.35 | 0.28 | 0.047 | 0.54 | 1.09 | 0.39 | 0.38 | 0.48 | 0.11 | 0.10 | 0.02 |
| 2 patients omitted from | −1.10 | 1.71 | 0.38 | −0.80 | 0.63 | 0.47 | 1.25 | 2.51 | 0.39 | 1.04 | 1.15 | 0.11 | 0.09 | 0.03 |
| analysis | | | | | | | | | | | | | | |

| | Change values of week 9 from baseline | | | Change values of week 11 from week 9 | | |
|---|---|---|---|---|---|---|
| C. All Cohort combined | Mean | SD | P-value* | Mean | SD | P-value* |
| Viral load PCRI^{H} | −0.50 | 0.96 | 0.03 | 0.04 | 0.73 | 0.81 |
| With 2 patients omitted from analysis | −0.71 | 1.72 | 0.10 | 0.17 | 1.76 | 0.69 |
| Viral load PCR II^{H} | −0.51 | 0.83 | 0.03 | 0.09 | 0.79 | 0.62 |
| With 2 patients omitted from analysis | −0.72 | 1.28 | 0.01 | 0.31 | 1.89 | 0.51 |

^{H}Log transformed Polymerase Chain reaction values, PCRI = all measures; PCRII = only viral load less than 400 copies/ml;
*Two-sided paired t-test;
**Ancova: adjusted for baseline value.

The results are presented using two approaches: first all measurements obtained by the more sensitive assay were used, even if they were over 400, and second, a second analysis was performed using only values from the more sensitive assay, if the new value was less than 400. Analysis of data from the more sensitive assays revealed no significant differences across treatment groups (p=0.23 for update I, and p=0.10 for update II), as well as no significant linear trend across dose levels (p=0.78 for update I and p=0.44 for update II). All four groups exhibited decreases in mean viral load. Comparison of mean changes from week 9 to week 11 (i.e., the post drug administration period), showed that there was a rebound effect seen at the two higher dose groups (C and D) using the more sensitive assay as noted by the significant linear trend (p=0.02 for update I, p=0.01 for update II, Table 1b). As shown in Table 1c, which compares mean changes for all dose groups combined, there was a statistically significant decrease in mean viral load using the more sensitive assays (p=0.03 for update I, p=0.01 for update II). The original viral load measures also showed a more modest decrease that did not reach statistical significance (p=0.22). No rebound effect was noted (p>0.62 for all three analyses). Because two patients changed their antiretroviral therapy during the study, there were some chances that these two patients contributed excessively to the viral load changes seen. Analyses were redone with these two patients omitted. Again in the baseline to week 9 analysis across doses, most groups had a decrease in viral load. Also, from week 9 to week 11 viral load increased, the greatest increase being in the highest doses groups. In conclusion there was a reduction of viral load of about one half log in all groups in the baseline to week 9 analysis. Interruption of drug treatment resulted in a rebound at the two higher doses.

C. Discussion

Procaine (SP01), Procainamide (SP100) and SP010 reduce HIV-1 IIIB replication in human cells with an efficacy higher than AZT, ddI or 3TC. In an experimental protocol without pre-medication, an inhibition of HIV-1 IIIB replication by these compounds was observed up to 50% with concentrations in the nanomolar range and there was not a major difference between the compounds dissolved in water compared to those dissolved in the Anticort formulation (SP01A, SP010A and SP100A). Surprisingly, within the range of 1 nM to 1 μM, SP010 displayed a higher efficacy than ddI in inhibiting viral replication.

In order to assess whether the virus was the direct target of the compounds or another mechanism is mediating the effect of these compounds on viral replication, the HeLa cells were pre-medicated for 24 hours with the different compounds in Anticort-like solution before the virus was added. Interestingly, the effect obtained was much stronger than without pre-medication and with concentrations in the picomolar range. The curve plateau was at more than 63% inhibition for SP01A, 52% for SP010A whereas it was around 32% for AZT. SP100A was less effective than AZT. In addition, the anti-viral activity of SP010A peaked up to 65% inhibition of the replication at 30 pM, and below 60% for SP01A whereas at the same concentration the inhibitory effect of AZT did not reach 30%.

Preincubation of the cells with the compounds under investigation for a 48 hours time period had even more pronounced effects, up to 80% inhibition of viral replication, even at picomolar concentrations. This difference in efficacy displayed after pre-medication versus no pre-medication suggests that the compounds under investigation may not directly target the virus but, more likely, modify the sensitivity of the cells to the virus entry, rendering them more resistant. Several observations established that inhibitors of cholesterol synthesis inhibit cell fusion formation induced by HIV-1 (Srivinas et al., AIDS Res Hum retrovir, 1994 10: 1489-1496) and that drugs extracting cholesterol from the cellular membrane exert an anti-HIV effect in vitro (Sarin et al., N Engl J Med, 1985 313: 1289-1290; Liao et al., AIDS Res Hum retrovir, 2001 17: 1009-1019; Maccarrone et al., J Neurochem, 2002 82(6): 1444-1452). In addition, it has been demonstrated that pre-incubation of procaine decreased the cholesterol synthesis rate limiting HMG-CoA mRNA expression induced by hormonal stimulation in mice and human adrenal cells (Xu et al., J Pharmacol Exp Therap, 2003 307:1147-1157).

These data suggest that procaine and procaine based compounds containing or derived from the SP01, SP010 and SP100 compounds reduce the HIV virus replication by modifying the cholesterol content of the cell membrane, rendering it much more difficult, even impossible, for the virus to entry and infect the cell. If this is true then it is believed that, in contrast to the classical anti-viral agents, such AZT, 3TC and ddI, SP01, SP10 and SP100 should be effective in blocking the HIV MDR 769 virus replication, due to reduced infectivity of the cells. Indeed, although AZT was ineffective in blocking HIV MDR 769 virus replication, SP01, SP010 and SP100 effectively blocked the replication of the virus/infectivity of the cells.

In a clinical setting, administration of procaine (SP01) in the Anticort formulation (SP01A) also caused a significant decrease in viral load of about 0.5 log between baseline and study end in patients under HAART therapy. The determination of viral load was made using a more sensitive assay, which compares favorably with many current NRTI medications.

In conclusion, the data herein demonstrates the ability of procaine, procainamide and the benzamide derivative SP010 to provide new anti-retroviral therapy efficaciuous either alone or in combination with HAART and mega HAART therapies. These results suggest that these compounds act most likely on mammalian cells by increasing their resistance to the virus entry rather than acting directly on the virus itself. Although the mechanism of action is not fully understood, an agent that acts on the host cells rather than directly on the virus can lower the rate of emergence of resistant strains and therefore to increase the efficacy of the current anti-retroviral therapies. The addition of oral procaine HCl in the Anticort formulation to the stable triple antiretroviral regimen of HIV+ patients demonstrated a reduction of viral load and an improvement in patient quality of life after just 9 weeks treatment. The finding that procaine in Anticort reduced the viral load in patients under HAART therapy, where viral load is supposed to be maximally suppressed, is in agreement with the in vitro studies presented above and indicates that the family of compounds disclosed in the present invention are beneficial in cases of resistance to triple antiretroviral therapy in HIV+ patients.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A method of treating a mammal exposed to a pathogen comprising contacting said cells with an effective amount of a compound of formula (I): 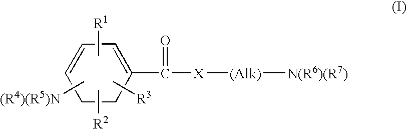
wherein:
a) R¹, R² and R³ are individually H, OH, halo, (C -C₆)alkyl, (C₁-C₆)alkoxy, (C₃-C₆)cycloalkyl, (C₃-C₆)cycloalkyl((C₁-C₆)alkyl), (C₂-C₆)alkenyl, (C₂-C₆)alkynyl, (C₁-C₆)alkanoyl, halo(C₁-C₆)alkyl, hydroxyl(C₁-C₆)alkyl, (C₁-C₆)alkoxycarbonyl; (C₁-C₆)alkylthio or (C₁-C₆)alkanoyloxy; or R¹ and R² together are methylenedioxy;
b) R⁴, R⁵, R⁶ and R⁷ are individually, H, (C₁-C₆)alkyl, (C₃-C₆)cycloalkyl, (C₃-C₆)cycloalkyl((C₁-C₆)alkyl), (C₂-C₆)alkenyl, wherein cycloalkyl optionally comprises 1-2, S, nonperoxide O or N(R⁵); aryl, aryl(C₁-C₆)alkyl, aryl(C₂-C₆)alkenyl, heteroaryl, heteroaryl(C₁-C₆)alkyl, or R⁴ and R⁵ or R⁶ and R⁷ together with the N to which they are attached form a 5- or 6-membered heterocyclic or heteroaryl ring, optionally substituted with R¹ and optionally comprising 1-2, S, non-peroxide O or N(R⁵);
c) (Alk) is (C₂-C₆)alkyl, (C₂-C₆)alkenyl, (C₃-C₆)cycloalkyl, (C₃-C₆)cycloalkyl(C₂-C₆)alkyl or [(C₂-C₆)alkyl(C₃-C₆)cycloalkyl[(C₃-C₆)alkyl] optionally substituted by 1-2 S, non-peroxide O or N(R⁵); and
d) X is O or NH;
and the pharmaceutically acceptable salts thereof.

2. The method of claim 1 wherein the amount is effective to inhibit entry of the pathogen or a subunit thereof into the cells.

3. The method of claim 1 wherein the pathogen is a virus.

4. The method of claim 1 wherein the pathogen is a retrovirus.

5. The method of claim 1 wherein the pathogen is HIV.

6. The method of claim 1 wherein the cells are contacted in vitro.

7. The method of claim 1 wherein the cells are contacted in vivo.

8. The method of claim 7 wherein the compound of formula I is administered to a human.

9. The method of claim 8 wherein the human has been exposed to a virus.

10. The method of claim 8 wherein the human has been exposed to a retrovirus.

11. The method of claim 10 wherein the human is HIV-positive.

12. The method of claim 10 wherein the human is an AIDS patient.

13. The method of claim 1 wherein (Alk) is (C₂-C₄)alkyl.

14. The method of claim 1 wherein both of R⁴ and R⁵ is H.

15. The method of claim 1 wherein both R⁶ and R⁷ are (C₁-C₆)alkyl or (C₃-C₆)cycloalkyl.

16. The method of claim 15 wherein both R⁶ and R⁷ are (C₁-C₄)alkyl or cyclohexyl.

17. The method of claim 1 wherein 1 or 2 of R¹, R² or R³ is (C₁-C₆)alkoxy.

18. The method of claim 17 wherein 1 or 2 of R¹, R² or R³ is (C₁-C₃)alkoxy.

19. The method of claim 1 wherein X is O.

20. The method of claim 1 wherein X is NH.

21. The method of claim 1 wherein the compound of formula I is administered orally.

22. The method of claim 1 wherein the compound of formula I is administered parenterally.

23. The method of claim 22 wherein the compound of formula I is administered by injection, infusion, inhalation or insufflation.

24. The method of claim 1 wherein the compound of formula (I) is administered in combination with a pharmaceutically acceptable carrier.

25. The method of claim 24 wherein the carrier is a liquid.

26. The method of claim 25 wherein the liquid is a solution, suspension or gel.

27. The method of claim 24 wherein the carrier is a solid.

28. The method of claim 24 wherein the carrier comprises zinc sulfate heptahydrate.

29. The method of claim 1 wherein the compound of formula I is procaine-HCl or procainamide-HCl.
